(19) United States
(12) Patent Application Publication
(10) Pub. No.: US 2023/0114990 A1
(43) Pub. Date: Apr. 13, 2023

(54) METHOD FOR FORMING COATED HYDROGEL BEADS

(71) Applicant: Oxford University Innovation Limited, Oxford(Oxfrodshire (GB)

(72) Inventor: Romain GUYON, Oxford (Oxfordshire) (GB)

(21) Appl. No.: 17/914,936

(22) PCT Filed: Mar. 29, 2021
(86) PCT No.: PCT/GB2021/050772

§ 371 Date: Sep. 27, 2022

(30) Foreign Application Priority Data
Mar. 27, 2020 (GB) 2004515.9

(51) Int. Cl. B01J 13/22; A61K 8/11; A61K 9/16; A23P 10/30

(52) U.S. Cl. CPC B01J 13/22; A61K 8/11; A61K 9/16; A23P 10/30

(57) ABSTRACT

The present invention provides a method of forming a coated hydrogel bead, wherein the hydrogel bead is coated via microfluidics.

The present invention relates to novel methods of making coated hydrogel beads, to coated hydrogel beads, and their use in controlled release of substances.

More specifically, the invention relates to methods of making coated hydrogel beads, where the coated hydrogel beads are made with precise control over physical characteristics such as bead size and coating thickness. The coated hydrogel beads made by the methods of the invention can be made within a narrow size range, and can encapsulate substances with consistently high encapsulation loading and minimal exposure of the encapsulated agent to organic solvent.

Current methods of encapsulating substances such as drugs and cosmetics include formation of double emulsions. In a double emulsion, droplets of a dispersed phase contain one or more types of smaller dispersed droplets themselves.

This double emulsion technique has many drawbacks, including formation of micro-particles with broad size ranges, inconsistent loading, and prolonged organic solvent exposure. These existing techniques lack precise control over physical particle characteristics including core size and polymer-shell thickness, all of which are key parameters for micro-particle controlled release systems.

The present invention seeks to address these problems and to provide a method of producing hydrogel beads with a more homogeneous and reproducible size range, an excellent encapsulation efficiency, and the ability to tailor release kinetics on demand with relative ease and without the need to validate for each different formulation.

The present inventors have found that hydrogel beads can be produced or coated using a single emulsion microfluidic device, via a convenient and readily scalable method. Hydrogel beads formed via one microfluidics single emulsion device can be injected into another, inversed, single emulsion system to add a coating layer around the bead, resulting in a distinct bead (“core”) and coating (“shell”).

The present inventors have thus found that hydrogel beads can be coated via an emulsion-based method which can produce coated beads of targeted and reproducible size ranges. The coating method may be combined with an emulsion-based method for bead formation, which also facilitates the production of beads with good control over size and homogeneity.

The formation and coating methods are compatible such that they allow beads to be formed, optionally isolated, e.g. for analysis, and subsequently coated, by performing the bead formation and coating methods in series. As the method lends itself to a variety of bead and coating materials, selection of the composition of the emulsion phases provides the ability to tailor the bead “core” and the coating (shell) characteristics for the desired end use.

Viewed from a first aspect, the present invention provides a method for coating hydrogel beads, i.e. a method for forming coated hydrogel beads, said method comprising:
forming an emulsion comprising a carrier fluid and hydrogel beads as a discontinuous phase, and an emulsifying fluid as a continuous phase; and
forming a coating on said hydrogel beads from the carrier fluid;
wherein said carrier fluid comprises a coating agent and the emulsifying fluid is immiscible with the carrier fluid.

A mixture of carrier fluid and hydrogel beads is used in the formation of the emulsion mentioned above. Providing this mixture may comprise forming hydrogel beads using an emulsion method as described herein. Methods for forming hydrogel beads via an emulsion method form a further aspect of the invention, preferably where the beads are subsequently coated via the methods described herein (e.g. the beads formed are used in the emulsion step above).

Thus, a further aspect of the present invention provides a method of forming coated hydrogel beads, said method comprising:
forming an emulsion (e.g. a first emulsion) comprising a hydrogel precursor composition as a discontinuous phase, and an emulsifying fluid (e.g. a first emulsifying fluid) as a continuous phase; and
forming hydrogel beads from the hydrogel precursor composition;
wherein the emulsifying fluid is immiscible with the hydrogel precursor composition. The beads formed are then coated, preferably by the method described herein.

Viewed from a further aspect, the invention provides a method for forming coated hydrogel beads (e.g. a method as described herein) wherein the hydrogel beads are coated via microfluidics.

A further aspect of the present invention provides a method for forming coated hydrogel beads wherein the beads are formed by, and coated by, an emulsion method, preferably a microfluidic emulsion method. Suitable methods for bead formation and bead coating are described herein. The beads may be coated subsequent to being formed, i.e. directly after being formed, for example formed in one microfluidic chip and then transferred, e.g. directly, to a second chip for the coating step.

Thus, in one embodiment, the present invention provides a method for forming coated hydrogel beads, said method comprising:
forming a first emulsion comprising a hydrogel precursor composition as a discontinuous phase and a first emulsifying fluid as a continuous phase; wherein the first emulsifying fluid is immiscible with the hydrogel precursor composition;
forming hydrogel beads from the hydrogel precursor composition;
forming a second emulsion, said second emulsion comprising a carrier fluid and the hydrogel beads as a discontinuous phase and a second emulsifying fluid as a continuous phase, wherein the carrier fluid comprises a coating agent and the second emulsifying fluid is immiscible with the carrier fluid; and
forming a coating on said hydrogel beads from the carrier fluid.

In some embodiments, the beads may be combined with the carrier fluid prior to formation of the second emulsion, i.e. the method comprises a step of combining the hydrogel beads with the carrier fluid to provide a mixture of carrier fluid and hydrogel beads. In such embodiments, the carrier fluid may be viewed as a coating fluid as herein described, where the coating fluid comprises the coating agent. References to “carrier fluid” and “coating fluid” used herein may be interchangeable unless suggested otherwise.

In other embodiments, the first emulsifying fluid comprises a coating agent, and thus a separate coating fluid is not required. In such embodiments, a step of mixing a coating fluid with the hydrogel beads is not necessary, as the beads can be formed in the first emulsifying fluid and the resulting mixture of beads and first emulsifying fluid can then be used to form the second emulsion (i.e. the step of forming the beads in the first emulsifying fluid provides the mixture of hydrogel beads and carrier fluid). In such embodiments, the first emulsifying fluid may be viewed as the carrier/coating fluid or vice versa, i.e. the first emulsifying fluid is involved in the first emulsification in the bead formation method and is also involved in the coating method.

The term “hydrogel” is used herein would be understood by the reader skilled in the art, and includes polymeric materials which exhibit the ability to swell in water and to retain a significant portion of water within their structure without dissolving.

The discontinuous phases of the emulsions herein described may comprise droplets, preferably microdroplets. In a preferred aspect of the present invention, the beads mentioned herein are microbeads. The terms “microdroplet” and “microbead” would be understood by the person skilled in the art and are intended to include the dimensions mentioned herein. For example, microdroplets may be 10 μm to 100 μm in diameter across the largest dimension and microbeads may be 2 μm to 500 μm in diameter across the largest dimension.

The term “(micro)droplet” is intended to encompass both “droplet” and “microdroplet”. Similarly, the term (micro)bead is intended to encompass both “bead” and “microbead”.

In some embodiments, the (micro)beads are crosslinked (micro)beads.

In one embodiment, the invention relates to a method of forming a coated hydrogel bead, wherein the hydrogel bead is formed via, and/or coated via, microfluidics, e.g. using a microfluidic chip or device.

In one embodiment, the invention relates to use of microfluidics (e.g. use of a microfluidic chip or device) in a method of forming a coated hydrogel bead.

As used herein, “microfluidics” refers to methods in which the behaviour and manipulation of fluids are geometrically constrained to a small scale (typically sub-millimeter), e.g. a scale at which capillary penetration governs mass transport.

In one embodiment, the invention relates to a method of forming a coated hydrogel bead wherein the hydrogel bead is formed using microfluidics (e.g. using a microfluidic chip or device) prior to coating. In one embodiment, the invention relates to a method of forming a coated hydrogel bead wherein a hydrogel bead is formed by forming an emulsion of a hydrogel precursor composition and an emulsifying fluid. The bead formation is carried out via a microfluidics method, e.g. in a microfluidics device. The hydrogel bead can then be coated.

In some embodiments, the present invention comprises a method of forming coated hydrogel beads said method comprising:
forming an emulsion, e.g. a first emulsion, comprising a hydrogel precursor composition as a discontinuous phase and an emulsifying fluid (e.g. a first emulsifying fluid) as a continuous phase; and
forming hydrogel beads from the hydrogel precursor composition; and
coating the hydrogel beads, e.g. via the method described herein;
wherein the first emulsifying fluid is immiscible with the hydrogel precursor composition.

In some embodiments, the method comprises:

(i) providing, (e.g. as a first phase), a hydrogel precursor composition;

(ii) providing, (e.g. as a second phase), an emulsifying fluid, wherein the emulsifying fluid is immiscible with the hydrogel precursor composition;

(iii) forming an emulsion, said emulsion comprising the hydrogel precursor composition as a discontinuous phase and the emulsifying fluid as a continuous phase;

(iv) forming hydrogel (micro)beads from the hydrogel precursor composition; and

(v) coating the hydrogel (micro)beads, e.g. via the method described herein.

A further aspect of the present invention provides a method of forming coated hydrogel beads, said method comprising:
contacting a hydrogel precursor composition with an emulsifying fluid (e.g. a first emulsifying fluid) wherein the emulsifying fluid is immiscible with the hydrogel precursor composition); and
forming hydrogel beads from the hydrogel precursor composition.

The beads formed are then coated, preferably by the method described herein.

The step of providing the hydrogel precursor composition may comprise flowing it through a microfluidic channel, and the step of providing the emulsifying fluid may comprise flowing it through a different microfluidic channel. The step of forming the emulsion may comprise contacting the hydrogel precursor composition with the emulsifying fluid (i.e. the two phases) at an intersection of the two (or more) microfluidic channels.

The step of forming the (first) emulsion may comprise contacting the hydrogel precursor composition with the (first) emulsifying fluid, preferably in a microfluidic channel, e.g. at an intersection of two (or more) microfluidic channels.

The emulsion may comprise (micro)droplets of hydrogel precursor composition dispersed in the emulsifying fluid.

The step of forming the emulsion may comprise bringing the phases into contact at an intersection of two microfluidic channels, e.g. such that the hydrogel precursor composition forms (micro)droplets within the emulsifying fluid.

In some embodiments, the step of forming the emulsion comprises injecting one phase into the other, e.g. by injecting the hydrogel precursor composition into a microfluidic channel comprising the emulsifying fluid. Preferably, the hydrogel precursor composition is injected microfluidically, i.e. using microfluidics.

In a preferred embodiment, the hydrogel (micro)beads are formed in the emulsion, i.e. in situ while the (micro)droplets are in emulsion with the emulsifying fluid.

For example, a method of forming a coated hydrogel bead according to the present invention may comprise:
(A) bringing a hydrogel precursor composition into contact with an emulsifying fluid (e.g. a first emulsifying fluid) in a channel, wherein the emulsifying fluid is immiscible with the hydrogel precursor composition;
(B) forming a hydrogel bead from the hydrogel precursor composition; and
(C) coating the hydrogel bead.

The hydrogel precursor composition may be brought into contact with the emulsifying fluid by injecting the hydrogel precursor composition into the emulsifying fluid. In some embodiments, the hydrogel precursor composition may be brought into contact with the emulsifying fluid at an intersection of two microfluidic channels.

The methods herein involve involving bringing at least two immiscible fluids or phases into contact, e.g. at a microfluidic intersection, such that one fluid will form a droplet inside the other fluid. Droplets, e.g. micro-droplets, are thus obtained. For example, in the steps of forming the bead, droplets are formed by bringing the hydrogel precursor composition into contact with the first emulsifying fluid, e.g. at the junction of a microfluidic chip. This forms an emulsion comprising droplets of hydrogel precursor composition with the emulsifying fluid as the continuous phase. The discontinuous phase of the first emulsion therefore comprises droplets (of the hydrogel precursor composition) which are formed into hydrogel beads.

The emulsifying fluid is immiscible with the hydrogel precursor composition, and vice versa, such that droplets (e.g. micro-droplets) of the hydrogel precursor composition are dispersed in the emulsifying fluid. This may be viewed as formation of an emulsion, e.g. a first emulsification and first emulsion. Similarly, the emulsifying fluid which is used to form the hydrogel beads prior to coating may be viewed as the first emulsifying fluid.

The emulsifying fluid used in the method for forming the beads may be referred to as the “first emulsifying fluid” where the emulsifying fluid used in the coating step may be referred to as the “second emulsifying fluid”.

The hydrogel precursor composition comprises a hydrogel precursor, optionally in combination with other components as herein described. The hydrogel precursor can be selected from any known hydrogel precursors, i.e. materials/compounds which can form hydrogels. Typically, these precursors are monomers or polymers that can be polymerised, gelled or cross-linked into hydrogel networks, e.g. crosslinkable compounds, preferably precursors to crosslinked hydrophilic polymers. Suitable precursors include polysaccharides (e.g. natural polysaccharides), proteins (e.g. natural proteins), monomers (e.g. synthetic monomers), polyacrylates, PEG (polyethylene glycol) and PEG derivatives, poloxamers, or PVA (polyvinyl alcohol) and combinations thereof.

In some examples, the polysaccharide is a crosslinkable polysaccharide.

Examples of natural polysaccharides include dextran, xanthan and carrageenan, alginates, chitosan, hyaluronic acid and agarose.

Examples of natural proteins include collagen and gelatin.

Suitable monomers include those for forming polyacrylates, PEG (polyethylene glycol) and

PEG derivatives, poloxamers, or PVA (polyvinyl alcohol). Examples of synthetic monomers include NIPAM (N-isopropylacrylamide).

Examples of PEG derivatives include those containing functional groups such as thiol, acrylate, norbornene, vinyl sulfone, maleimide, furan, and acrylates, e.g. diacrylate.

PEG derivatives may include multi-arm PEG (e.g., 3, 4, 6, or 8-arm, or Y-shaped), and/or those with different functionalities at the extremities of the arms to achieve crosslinking, such as the thiol, acrylate, norbornene, vinyl sulfone, maleimide, furan, and acrylates, e.g. diacrylate, mentioned above. For example, multi-arm PEG derivatives include 8-arm PEG, 8-arm PEG norbornene (e.g. 20 mM [NB] groups), 8-arm PEG acrylate and 8-arm PEG thiol, and combinations thereof, e.g. a combination of 8-arm PEG acrylate and 8-arm PEG thiol, preferably a 1:1 combination of PEG derivatives such as 8-arm PEG acrylate and 8-arm PEG thiol.

Examples of poloxamers include Pluronic®, e.g. Pluronic® F127 or Pluronic® F68.

Preferred examples of hydrogel precursors include PEG and derivatives thereof, especially PEG acrylate, PEG norbornene, PEG thiol and agarose.

The hydrogel precursor composition can comprise the hydrogel precursor(s) in an amount of from 1 to 50% w/v, for example 10 to 50% w/v, e.g. 12 to 40% w/v, 15 to 35% w/v, 18 to 30% w/v, 20 to 25% w/v, based on the total volume of the hydrogel precursor composition. Particularly preferred amounts, in weight percent, are 1 to 30, e.g. 2 to 10, e.g. 3 to 7%.

The hydrogel precursor composition can consist of (e.g. consist essentially of) hydrogel precursor(s) (in which case the hydrogel precursor composition is the hydrogel precursor compound or material), or can comprise further components such as encapsulated agents, solvents (such as water, deionized water, buffers), additives (such as a photoinitiators, for example LAP, Lithium phenyl-2,4,6-trimethylbenzoylphosphinate and Irgacure® 2959), or linker molecules (e.g. crosslinkers) such as Ca²⁺ ions and dithiols (e.g. dithiothreitol)). In some embodiments, the hydrogel precursor composition further comprises one or more additional components selected from:
encapsulated agents,
solvents (such as water, deionized water, buffers),
additives (such as a photoinitiators, for example LAP, Lithium phenyl-2,4,6-trimethylbenzoylphosphinate and Irgacure® 2959), and
linker molecules (e.g. crosslinkers) such as Ca²⁺ ions and dithiols (e.g. dithiothreitol)).

If the hydrogel formation is to be initiated by UV, the hydrogel precursor composition can further comprise additives to ensure crosslinking, (such as a photoinitiator, for example LAP, Lithium phenyl-2,4,6-trimethylbenzoylphosphinate or Irgacure® 2959).

Additives can be present in the hydrogel precursor composition in an amount of from 1 to 20 mM, e.g. 1 to 10 mM, 3 to 8 mM or 5 to 15 mM, 8 to 13 mM, 10 to 12 mM. Typical amounts of additive, expressed in relation to the hydrogel precursor composition, are 0.25 to 5 w/v %, e.g. 0.5 to 3 w/v %.

The hydrogel precursor composition may comprise a linker molecule that will enable the formation of the network between the hydrogel precursor by chemical or physical reaction (e.g. Ca²⁺ ions for alginate, or a dithiol small linker (e.g. DTT, dithiothreitol, for example 40 mM DTT (80 mM [SH] group) for crosslinking of PEG via Michael Addition).

Linker molecules can be present in the hydrogel precursor composition in an amount of from 10 to 50% w/v, e.g. 12 to 40% w/v, 15 to 35% w/v, 18 to 30% w/v, 20 to 25% w/v, based on the total volume of the hydrogel precursor composition. The linker molecules may be present in the hydrogel precursor composition in equimolar amounts with respect to the hydrogel precursor.

The hydrogel bead formed by method of the invention can be a hydrogel bead formed from and/or comprising the afore-mentioned hydrogel precursors, especially crosslinked, gelled or polymerised versions thereof. Examples comprise hydrogels (e.g. crosslinked hydrophilic polymers) formed from and/or being or comprising polysaccharides (e.g. natural polysaccharides), proteins, (e.g. natural proteins), monomers (e.g. synthetic monomers), polyacrylates, PEG (polyethylene glycol) or PEG derivatives, poloxamers, Poly(N-isopropylacrylamide) or PVA (polyvinyl alcohol), e.g. those mentioned herein, or combinations thereof. In addition to the hydrogel component, the bead may additionally comprise the additives (e.g. encapsulated agents) mentioned herein.

The hydrogel bead may be spherical or non-spherical, preferably substantially spherical.

In the steps of bead formation, the (first) emulsifying fluid is a fluid which is immiscible with the hydrogel precursor composition. This immiscibility causes droplets (e.g. micro-droplets) of the hydrogel precursor composition to form in the emulsifying fluid. The nature of the emulsifying fluid may therefore be selected according to the hydrogel precursor composition. Typically, the (first) emulsifying fluid is a lipophilic fluid, e.g. an oil or a lipophilic polymer such as polylactic acid (PLA), poly(lactic-co-glycolic) acid (PLGA), polycaprolactone (PLC), polyanhydrides etc.

Examples of suitable emulsifying fluids include oils, HFE (hydrofluoroethers), FC-40 (Fluorinert® oil), light mineral oil, olive oil, hexadecane, sunflower seed oil, castor oil, surfactants, solvents (especially organic or lipophilic solvents), polymers (e.g. lipophilic polymers) and combinations thereof. Suitable surfactants include polyvinyl alcohol.

In some embodiments, materials suitable for use as the emulsifying fluid discussed herein in connection with the method for forming a hydrogel bead (e.g. the first emulsifying fluid) are also suitable for use as the carrier fluid, coating agent or coating fluid (and vice versa) in the coating steps. Suitable materials for use as the emulsifying fluid in the method for forming a hydrogel bead therefore include those disclosed herein as carrier fluid, coating agent or coating fluid, preferably those which are lipophilic. Examples include polymers, polymer precursors, monomers, solvents (especially organic or lipophilic solvents), or mixtures thereof.

In some embodiments, the polymer is a lipophilic polymer.

Examples of suitable polymers include polyesters and polyanhydrides. Examples of polyesters include poly(lactic-co-glycolic) acid (PLGA, e.g. PLGA R520), polylactic acid (PLA) and polycaprolactone (PLC). One or more polyesters can be present. If poly(lactic-co-glycolic) acid is used, the lactic/glycolic ratios can be in a ratio of lactic:glycolic of from 5:95 to 100:0, e.g. from 10:90 to 95:5, 15:85 to 90:10, 20:80 to 85:15, 25:75 to 80:20, 30:70 to 75:25, 35:65 to 70:30, 40:60 to 65:35, 45:55 to 60:40, 50:50 to 55:45. Examples of polyanhydrides include poly(sebacic acid).

The molecular weight of polymer, e.g. polyesters, used can be from 2 to 40 kDa, e.g. from 5 to 35 kDa, 10 to 30 kDa, 15 to 20 kDa, e.g. 7 to 17 kDa.

The emulsifying fluid may be or comprise (e.g. in addition to the other materials described herein) a solvent, e.g. a lipophilic solvent or an organic solvent.

When the emulsifying fluid comprises a polymer, e.g. a lipophilic polymer, it will typically additionally comprise a hydrophobic solvent or an organic solvent. Preferably, the emulsifying fluid comprises a solvent (preferably an organic solvent) and a polymer.

Suitable solvents include chloroform, dichloromethane (DCM), dimethylcarbonate (DMC), benzyl acetate, ethyl acetate, anisole, and mixtures thereof.

An example of a first emulsifying fluid which can also be used as a coating fluid in the coating method described herein is a mixture of PLGA and DMC, e.g. 10% w/w PLGA in DMC.

The emulsifying fluid may additionally comprise further components such the additives described herein, e.g. photoinitiators and/or linker molecules.

As noted above, the hydrogel precursor composition (and thus the resulting beads) may comprise one or more encapsulated agents. This is particularly preferred when the resulting coated hydrogel beads are intended for controlled release applications, because the encapsulated agent(s) can be loaded into the hydrogel beads for later release.

The amount of encapsulated agent in the bead (sometimes referred to as the degree of “loading”) can be tailored by the amount present in the hydrogel precursor composition, and will be selected according to the desired function of the encapsulated agent in the beads. The hydrogel precursor composition may comprise any encapsulated agent in a concentration of up to 80% by weight relative to the total amount of hydrogel precursor composition (i.e. relative to the total amount of precursor, active agent and any additional additives), e.g. 1 to 80 wt %, 2 to 75 wt %, 3 to 65 wt %, 5 to 65 wt %, 8 to 65 wt %. 10 to 60 wt %, 15 to 60 wt %, 20 to 60 wt %, 25 to 50 wt %, or 30 to 40 wt %, preferably up to 20 wt %, e.g. 1 to 18 wt % or 2 to 15 wt %, especially preferably up to 10 wt %, e.g. 1 to 10 wt %, 1 to 8 wt %, 1 to 5 wt % or 5 to 10 wt %.

Typically, any encapsulated agent(s) or other additives, are combined with the hydrogel precursor (and any other components of the hydrogel precursor composition) before bringing the hydrogel precursor composition into contact with the emulsifying fluid. The various components for the hydrogel precursor composition may be combined via any suitable method, e.g. mixing.

As used herein, “encapsulated agent” refers to an agent or substance that is encapsulated in the hydrogel bead. The encapsulated agent may be one intended for medical or non-medical uses.

Typically the encapsulated agent is a substance that is desired to be released at a time later than the administration of the substance in which it is encapsulated. The encapsulated agent is therefore preferably a substance for controlled release or sustained release in later use of the coated hydrogel bead. Any substance known for controlled-release applications and compatible with a hydrogel may be used. The encapsulated agent may have a specific and/or measurable effect, e.g. a physiological activity when administered to a subject in a pharmaceutically effective amount.

The term “encapsulated agent” as used herein encompasses agents or substances used in medicine (e.g. a therapeutic, a pharmaceutical, a prophylactic, or a diagnostic agent), the food industry, or the cosmetic industry. Examples include pharmaceutical compounds or compositions, nutritional supplements, flavours, minerals, vitamins, small molecule drugs, proteins, viruses (e.g. therapeutic viruses), vaccines, therapeutic antibodies, nutritional supplements, carotene, biomarkers, tracer dyes, fluorescent dyes and combinations thereof, preferably proteins, vaccines, therapeutic antibodies, viruses (e.g. therapeutic viruses), biomarkers, tracer dyes, fluorescent dyes and combinations thereof.

In some embodiments, the encapsulated agent may be one with a detectable property. The agent may be intended for delayed release and/or detection, e.g. for applications such as tracer dyes. Detectable agents may be used for medical or non-medical applications. Suitable encapsulated agents for these applications include dyes, e.g. fluorescent dyes such as Rhodamine (e.g. Rhodamine B or Dextran-Rhodamine B), Alexa Fluor dyes, fluorescent dyes covalently bonded to dextran, or fluorescent dye-dextran conjugates (e.g. Dextran-FITC, Dextran-TRITC, etc.).

The hydrogel precursor composition or the hydrogel bead may therefore comprise an encapsulated agent, preferably an encapsulated agent selected from pharmaceutical compounds or compositions, nutritional supplements, flavours, minerals, vitamins, small molecule drugs, proteins, therapeutic viruses, vaccines, therapeutic antibodies, nutritional supplements, carotene, biomarkers, tracer dyes, fluorescent dyes and combinations thereof.

The hydrogel precursor composition and (first) emulsifying fluid are brought into contact with each other, e.g. by causing streams of each to meet, preferably in a microfluidics device, e.g. by contacting them at an intersection of two microfluidic channels. The relative flow rates of the hydrogel precursor composition and emulsifying fluid in the method, e.g. microfluidics method, and/or the channel dimensions can be selected to control the droplet size (i.e. the size of the droplets of the discontinuous phase), which can in turn influence the size of the hydrogel bead formed. In instances where the hydrogel precursor is combined with an encapsulated agent, the flow rate and/or channel dimension can therefore be used to tailor the amount of encapsulated agent or ingredient present in each droplet.

Bringing the hydrogel precursor composition into contact with an emulsifying fluid in a channel can be achieved by:
1. flowing the hydrogel precursor composition through a first inlet channel;
2. flowing the emulsifying fluid though a second channel; and
3. bringing the hydrogel precursor composition and emulsifying fluid into contact, e.g. at a junction of the first and second inlet channel.

In some embodiments, the emulsions mentioned herein are formed using flow-focusing.

In some embodiments, bringing the hydrogel precursor composition into contact with the emulsifying fluid is achieved via injection of one into the other. In some embodiments, contact is achieved by bringing the hydrogel precursor composition into contact with the emulsifying fluid at an intersection of two or more microfluidic channels.

Causing the hydrogel precursor composition (e.g. the droplets of hydrogel precursor composition formed due to contact with the emulsifying fluid) to form a hydrogel bead can be achieved by any methods known in the art. Any method suitable forming a gel or solid, e.g. a cross-linked, gelled, or polymerised bead, from the precursor composition, e.g. from the hydrogel precursor, may be used. In some embodiments, the hydrogel precursor composition forms a hydrogel bead via UV exposure, temperature, ionic gelation or a chemical reaction.

Preferably, the beads are formed from the hydrogel precursor in the presence of the first emulsifying fluid, i.e. the droplets of hydrogel precursor composition are converted to hydrogel beads while dispersed in the first emulsifying fluid. This results in hydrogel beads in the first emulsifying fluid.

The droplets of hydrogel precursor composition may be collected from the channel, chip or device in which the emulsion is formed (e.g. from the microfluidic chip) into a vial/container while still in emulsion with, i.e. dispersed in, the emulsifying fluid, prior to formation of the bead/gel.

UV exposure can involve exposing the dispersion of hydrogel precursor to a suitable UV exposure regime, for example one selected relative to any photoinitiator used. Typical UV exposure steps include exposure to 10-400 nm wavelength electromagnetic radiation, e.g. 100-400 nm (such as 100-315 nm (e.g. 100-280 nm) or 280-400 nm (e.g. 315-400 nm)), preferably 250-380 nm, 255-340 nm, 280-315 nm, or 365 nm. UV exposure can be for 10 seconds to 60 min, e.g. 30 seconds to 30 min, 45 seconds to 10 min, 1 min to 5 min, e.g. 3 min. UV exposure, e.g. the UV source, can be from 2 to 30 cm from the dispersion of hydrogel precursor (i.e. the droplets in emulsion with the emulsifying fluid), e.g. from 5 to 25 cm, 10 to 20 cm, 12 to 18 cm, 15 to 22 cm from the dispersion of hydrogel precursor.

Use of temperature can involve exposing the dispersion of hydrogel precursor in the emulsifying fluid to temperatures below their gelation temperature or above their critical gelation temperatures, depending on the type of hydrogel selected. For example, when ultra-low temperature melting agarose is used, a temperature of around 8° C. can be used, and when poloxamers (such as Pluronic® F127 or Pluronic® F68 are used) temperatures above the critical gelation temperature can be used. These temperatures depend on concentration (for example, temperatures of around 30° C. for a 15% Pluronic® F127 precursor solution can be used).

Use of a chemical reaction can involve incubation to let the reaction occur (e.g. incubation from 1 min to 6 hours, e.g. 5 min to 4 hours, 30 min to 2 hours, 45 min to 1 hour). Reaction times can depend on the concentration and strength of any functional groups. For example, for a Michael addition (e.g. with PEGs between thiol groups and “ene” groups [C═C bonds]), or a Diels Alder reaction (e.g. a Diels-Alder reaction between PEG with maleimide functionalities and furan functionalities), the chemical reaction can require a time from 1 min to 6 hours, e.g. 5 min to 4 hours, 30 min to 2 hours, 45 min to 1 hour to crosslink.

It is also possible for the hydrogel precursor composition to be formed into a hydrogel bead by altering the pH. Altering the pH of the surrounding media of the hydrogel beads can catalyse the reaction, and depends on the functionality of the hydrogel bead, e.g. the composition of the hydrogel precursor composition. For example PEG acrylate and PEG thiol reacts by Michael addition in minutes to hours at pH 7, but reacts in seconds to minutes at pH 8 and above.

Ionic gelation, caused by diffusion of Ca²⁺ ions in the hydrogel, can also trigger formation of a hydrogel bead from a hydrogel precursor composition. For example, ionic gelation can be used to rapidly (i.e. in less than 1 hour from exposure to Ca²⁺ ions, e.g. less than 45 min, less than 30 min, less than 15 min, less than 10 min or less than 5 min) trigger alginate crosslinking by ionic interactions.

The step of forming a hydrogel bead from the droplets of hydrogel precursor composition therefore involves polymerising, gelling or crosslinking the hydrogel precursor. The liquid droplets of hydrogel precursor composition are thus formed into hydrogel beads. The hydrogel beads may be in solid or gel form.

Prior to coating, the hydrogel beads formed via the methods herein described, e.g. using microfluidics (e.g. those formed by steps (A) and (B) and other methods described herein) may be collected or isolated, e.g. separated from the (first) emulsifying fluid. Suitable methods include centrifugation, filtration and oil extraction. This allows the quality of the hydrogel beads to be assessed, and can result in increased quality of the coated hydrogel beads. Collection also enables a washing or rinsing step, if desired, e.g. to remove residual emulsifying fluid or components thereof.

In some embodiments, the beads may collected or isolated from the first emulsifying fluid, preferably by centrifugation, filtration or oil extraction, prior to being combined with the carrier fluid, especially when the carrier fluid is a coating fluid (i.e. the carrier fluid is different to the first emulsifying fluid).

In some embodiments, for example, when the first emulsifying fluid comprises a coating agent, and thus can be used as the carrier fluid in the coating method, the beads can be formed in the (first) emulsifying fluid, and then coated, e.g. without collection, isolation and/or washing.

Coating the hydrogel beads formed using microfluidics (e.g. those formed by steps (A) and (B) and other methods described herein) can be achieved using any method known in the art. In some embodiments, the hydrogel beads can be coated using microfluidics, electrospray deposition, acoustofluidics, spray drying, spin coating, bulk emulsification and combinations thereof. In a preferred embodiment, the beads are coated by the methods described herein, e.g. the hydrogel beads formed via microfluidics are then coated using microfluidics, e.g. via the microfluidic coating methods described herein.

After coating the hydrogel bead to form a coated hydrogel bead, the coated hydrogel beads can be collected prior to further use. The hydrogel beads can be collected using any methods of the art, including centrifugation, filtration and oil extraction.

In an embodiment, the invention relates to a method of coating hydrogel beads (e.g. hydrogel beads formed by the method described herein) via microfluidics, e.g. using a microfluidic device or chip. In an embodiment, the invention relates to a method of coating hydrogel beads (e.g. hydrogel beads formed by the method described herein), said method comprising forming an emulsion of a mixture of carrier/coating fluid and hydrogel bead(s) and an emulsifying fluid. A coating is formed from the carrier/coating fluid. The emulsion formation is carried out via a microfluidics method, e.g. in a microfluidics device.

Viewed from one aspect, the present invention provides a method for forming coated hydrogel (micro)beads, said method comprising:
forming an emulsion comprising a carrier fluid and hydrogel (micro)beads as a discontinuous phase, and an emulsifying fluid as a continuous phase; and
forming a coating on said hydrogel (micro)beads from the carrier fluid;
wherein said carrier fluid comprises a coating agent and the emulsifying fluid is immiscible with the carrier fluid.

The method may comprise, prior to forming the emulsion comprising the carrier fluid and hydrogel (micro)beads as a discontinuous phase, providing a mixture of the carrier fluid and hydrogel (micro)beads.

In some embodiments, a method for coating hydrogel (micro)beads (e.g. hydrogel (micro)beads formed by the methods described herein) may comprise:

(a) providing, (e.g. as a first phase), a mixture of a carrier fluid and hydrogel (micro)beads;

(b) providing, (e.g. as a second phase), an emulsifying fluid, wherein the emulsifying fluid is immiscible with the carrier fluid;

(c) forming an emulsion, said emulsion comprising the carrier fluid and hydrogel (micro)beads as a discontinuous phase and the emulsifying fluid as a continuous phase; and

(d) forming a coating on said hydrogel (micro)beads from the carrier fluid.

A further aspect of the present invention provides a method for coating hydrogel beads, i.e. a method for forming coated hydrogel beads, said method comprising:
contacting an emulsifying fluid with a mixture of a carrier fluid and hydrogel beads, wherein said carrier fluid comprises a coating agent and the emulsifying fluid is immiscible with the carrier fluid; and
forming a coating on said hydrogel beads from the carrier fluid.

The step of providing the mixture of the carrier fluid and hydrogel (micro)beads may comprise flowing the mixture through a microfluidic channel, and the step of providing the emulsifying fluid may comprise flowing it through a different microfluidic channel. The step of forming the emulsion may comprise contacting the mixture of carrier fluid and hydrogel (micro)beads with the emulsifying fluid (i.e. the two phases) at an intersection of the two (or more) microfluidic channels.

The step of forming the emulsion may comprise contacting the mixture of carrier fluid and hydrogel (micro)beads with the emulsifying fluid, preferably in a microfluidic channel, e.g. at an intersection of two (or more) microfluidic channels.

The emulsion comprises (micro)droplets of carrier fluid dispersed in the emulsifying fluid, wherein one or more of said (micro)droplets comprises a hydrogel (micro)bead.

The step of forming the emulsion may comprise bringing the phases into contact at an intersection of two (or more) microfluidic channels, e.g. such that the mixture of carrier fluid and hydrogel beads forms (micro)droplets within the emulsifying fluid.

Is some embodiments, the step of forming the emulsion comprises injecting one phase into the other, e.g. by injecting the mixture of carrier fluid and hydrogel beads into a microfluidic channel comprising the emulsifying fluid. Preferably, the mixture of carrier fluid and hydrogel beads is injected microfluidically.

The mixture of carrier fluid and hydrogel (micro)beads may be a suspension (e.g. of the beads in carrier fluid). Preferably, the beads should not be soluble in the carrier fluid, e.g. the hydrogel is preferably immiscible with the carrier fluid.

In preferred embodiments, the methods described herein are performed in a microfluidic system, chip, or device.

In a preferred embodiment, the coating is formed in the emulsion, i.e. in situ while the (micro)droplets containing the (micro)beads are in emulsion with the second emulsifying fluid as the continuous phase.

In some embodiments, the second emulsifying fluid (i.e. the emulsifying fluid used to form an emulsion in the coating method) is immiscible with both the carrier fluid and the hydrogel (micro)beads.

The carrier fluid comprises a coating agent as described herein. Preferably, the second emulsifying fluid (i.e. the emulsifying fluid used to form an emulsion in the coating method) is immiscible with the coating agent.

In some embodiments, the carrier fluid is a coating fluid as herein described and thus and the step of providing the mixture of carrier fluid and hydrogel (micro)beads comprises combining the hydrogel beads with the coating fluid.

For example, a method of coating hydrogel beads (e.g. hydrogel beads formed by the method described herein) may comprise:
(1) bringing a mixture of carrier/coating fluid and hydrogel bead(s) into contact with an emulsifying fluid in a channel, wherein the emulsifying fluid is immiscible with the coating fluid (and/or vice versa); and
(2) forming a coating from the carrier/coating fluid.

The mixture of carrier/coating fluid and hydrogel bead(s) may be brought into contact with the emulsifying fluid by injecting one into the other, e.g. by injecting the mixture of carrier/coating fluid and hydrogel beads into the emulsifying fluid in a channel.

In some embodiments, the mixture of carrier/coating fluid and hydrogel beads may be brought into contact at an intersection of two (or more) microfluidic channels.

The emulsifying fluid used in the coating method is immiscible with the carrier/coating fluid such that droplets of the carrier/coating fluid (e.g. micro-droplets) containing the hydrogel beads are dispersed in the emulsifying fluid. This may be viewed as formation of an emulsion, e.g., in embodiments where the hydrogel bead was itself formed in an emulsion process (e.g. a first emulsification), this could be viewed as a second emulsification and a second emulsion. The emulsifying fluid which is immiscible with the carrier fluid may therefore be considered to be the second emulsifying fluid.

In a further embodiment, the invention relates to a method of forming coated hydrogel beads, wherein the method of forming the hydrogel bead and the method of coating the hydrogel bead are both performed by microfluidics, e.g. using a microfluidic device or chip. Such a method may combine the methods for forming beads with the methods for coating beads herein described (e.g. involving steps (A) and (B) as discussed herein followed by steps (1) and (2) as discussed herein).

For example, the method of forming coated hydrogel beads may comprise:
bringing a hydrogel precursor composition into contact with an emulsifying fluid (e.g. a first emulsifying fluid) in a channel, wherein the (first) emulsifying fluid is immiscible with the hydrogel precursor composition;
forming a hydrogel bead from the hydrogel precursor composition (and optionally collecting or isolating the hydrogel beads);
bringing a mixture of coating fluid and the hydrogel beads into contact with an emulsifying fluid (e.g. a second emulsifying fluid) in a channel (e.g. wherein the second emulsifying fluid is immiscible with the coating fluid); and
forming a coating from the coating fluid.

A further aspect of the present invention provides a method for forming coated hydrogel beads, said method comprising:
contacting a hydrogel precursor composition with a first emulsifying fluid, wherein the first emulsifying fluid is immiscible with the hydrogel precursor composition;
forming hydrogel beads from the hydrogel precursor composition;
contacting a second emulsifying fluid with a mixture of a carrier fluid and the hydrogel beads, wherein the carrier fluid comprises a coating agent and the second emulsifying fluid is immiscible with the carrier fluid; and
forming a coating on said hydrogel beads from the carrier fluid.

In another embodiment, the present invention provides, a method for forming coated hydrogel (micro)beads, said method comprising:
providing a hydrogel precursor composition;
providing a first emulsifying fluid, wherein the first emulsifying fluid is immiscible with the hydrogel precursor composition;
forming a first emulsion, said first emulsion comprising the hydrogel precursor composition as a discontinuous phase and the first emulsifying fluid as a continuous phase;
forming hydrogel (micro)beads from the hydrogel precursor composition;
combining the hydrogel (micro)beads with a coating fluid to provide a mixture of coating fluid and hydrogel (micro)beads,
providing a second emulsifying fluid, wherein the second emulsifying fluid is immiscible with the coating fluid;
forming a second emulsion, said second emulsion comprising the coating fluid and hydrogel bead as a discontinuous phase and the second emulsifying fluid as a continuous phase; and
forming a coating on said hydrogel (micro)beads from the coating fluid.

Without being bound by theory, it is thought that using a separate method for coating hydrogel beads (to that used to produce the hydrogel beads) can help the breakup of the droplets by the induction of a Rayleigh-Plateau instability. This may increase the flow rate that can be used in the method, increasing the output.

Using a separate method for coating hydrogel beads may also allow analysis of the hydrogel beads prior to coating. This allows the quality of the hydrogel beads to be assessed, and can result in increased quality of the coated hydrogel beads.

A further effect of using a separate method for coating hydrogel beads, is that it allows for the mass production of hydrogel beads. These hydrogel beads can then be coated using different coatings, resulting in the formation of identical core particles with different shell composites (e.g. varying in shell thickness, polymer composition or polymer families). This means that release kinetics can be tailored on demand with relative ease and without the need to validate for each different formulation.

A person skilled in the art would appreciate that formation of droplets of carrier/coating fluid which contain hydrogel beads (e.g. the formation of such droplets as outlined in step (1) of the method of coating hydrogel beads as discussed above) is governed by Poisson distribution. It is therefore possible to accurately predict the number of droplets that are unloaded and loaded with one or more hydrogel beads, depending on the concentration of the hydrogel beads in the carrier/coating fluid. Typically, a droplet contains one bead.

In addition to reliance on the Poisson distribution, there are many other methods in the art which can be used to provide efficient encapsulation of hydrogel beads in droplets of carrier/coating fluid. For example, lasers can be used to guide particles, inertial ordering can be used to passively organise particles prior to encapsulation, or particles can be close packed and encapsulated by matching the periodicity of the drop formation to the particle flow (see Abate et al, Lab Chip, 2009, 9, 2628).

Bringing the coating fluid into contact with the emulsifying fluid in a channel can be achieved by:
1. flowing the mixture of coating fluid and hydrogel beads through a first inlet channel;
2. flowing the emulsifying fluid though a second channel; and
3. bringing the mixture of coating fluid and hydrogel beads into contact with the emulsifying fluid, e.g. at a junction of the first and second inlet channel.

The carrier fluid comprises a coating agent. The carrier fluid may be a coating fluid as described herein. Alternatively, in embodiments where the first emulsifying fluid comprises a coating agent, the carrier fluid may be, or comprise, the first emulsifying agent.

The carrier/coating fluid is, consists of, consists essentially of, or comprises, one or more coating agents. The coating agent is a material capable of forming, or being converted into, a coating on the hydrogel beads disclosed herein, e.g. it may be a coating precursor. Typically, the coating agent is a material capable of forming a coating suitable for controlled release applications. In some embodiments, the coating agent is a material capable of forming, or being converted into, a coating with a degree of permeability. The coating agent may be selected according to the characteristics desired for the coated beads.

The coating agent can be, or comprise, a polymer, a polymer precursor, a monomer, a pre-gel, or mixtures thereof.

Non-liquid polymers may be combined with a suitable solvent to form the carrier fluid. In some embodiments, the polymer is a lipophilic polymer, which may be combined with an organic or lipophilic solvent to form the carrier fluid.

Examples of suitable polymers include polyesters and polyanhydrides.

Examples of suitable pre-gels include the hydrogel precursors as discussed herein, e.g. 8-arm PEG acrylate and/or 8-arm PEG thiol, e.g. in equimolar concentrations of acrylate and thiol.

Examples of polyesters include poly(lactic-co-glycolic) acid (PLGA, e.g. PLGA R520), polylactic acid (PLA) and polycaprolactone (PLC). One or more polyesters can be present. If poly(lactic-co-glycolic) acid is used, the lactic/glycolic ratios can be in a ratio of lactic:glycolic of from 5:95 to 100:0, e.g. from 10:90 to 95:5, 15:85 to 90:10, 20:80 to 85:15, 25:75 to 80:20, 30:70 to 75:25, 35:65 to 70:30, 40:60 to 65:35, 45:55 to 60:40, 50:50 to 55:45.

The molecular weight of polymer, e.g. polyesters, used can be from 1 to 150 kDa, preferably 2 to 40 kDa, e.g. from 5 to 35 kDa, 10 to 30 kDa, 15 to 20 kDa, e.g. 7 to 17 kDa.

Examples of polyanhydrides include poly(sebacic acid).

In addition to the coating agent, the carrier/coating fluid may additionally comprise further components such as solvents (e.g. a lipophilic solvent or an organic solvent), additives, or mixtures thereof. When the coating agent is, or comprises a polymer, e.g. a lipophilic polymer, the carrier/coating fluid will typically additionally comprise an organic or lipophilic solvent. When the coating agent is, or comprises a pre-gel, the coating fluid will typically additionally comprise an aqueous solvent (e.g. the coating fluid comprises a pre-gel and an aqueous solvent).

Examples of suitable additives include photoinitiators, (for example LAP, lithium phenyl-2,4,6-trimethylbenzoylphosphinate and Irgacure® 2959), and linker molecules (such as Ca²⁺ ions and dithiols (e.g. dithiothreitol)).

Preferably, the carrier/coating fluid comprises a solvent (preferably an organic solvent or a hydrophobic solvent) and a polymer (preferably a lipophilic polymer).

Examples of suitable organic solvents include chloroform, dichloromethane (DCM), dimethylcarbonate (DMC), benzyl acetate, ethyl acetate, anisole, and mixtures thereof.

Examples of suitable aqueous solvents include water (e.g. deionized water) and aqueous buffers (such as phosphate buffer, PBS).

In some embodiments, materials suitable for use as the emulsifying fluid discussed herein in connection with the method for forming a hydrogel bead (e.g. the first emulsifying fluid) are also suitable for use as the carrier/coating fluid (and vice versa).

The carrier/coating fluid may therefore be or comprise any of the materials described herein in connection with the first emulsifying fluid, e.g. including lipophilic fluids, oils, HFE (hydrofluoroethers), FC-40 (Fluorinert® oil), light mineral oil, olive oil, hexadecane, sunflower seed oil, castor oil, surfactants, solvents (especially organic or lipophilic solvents), polymers (e.g. lipophilic polymers), and combinations thereof. Suitable surfactants include polyvinyl alcohol.

In some embodiments, the same composition can be used as the emulsifying fluid discussed herein in connection with the method for forming a hydrogel bead (e.g. the first emulsifying fluid) and as coating agent or carrier/coating fluid. In some embodiments a mixture of a polymer and a solvent is used as the first emulsifying fluid and as the carrier/coating fluid.

An example of an emulsifying fluid which can also be used as a coating fluid in the coating method described herein is a mixture of PLGA and DMC, e.g. 10% w/w PLGA in DMC.

Especially perfectly, the carrier fluid comprises a lipophilic polymer and a hydrophobic solvent.

Where the first emulsifying fluid is compatible with the coating agent/fluid, e.g. if the first emulsifying fluid has the same (or similar) composition as the coating agent or fluid, it may not be necessary to collect and wash the beads following polymerisation/gelation and prior to mixing them with the coating fluid.

In one embodiment, the first emulsifying fluid is selected such that it comprises a coating agent, while also being immiscible with the hydrogel precursor composition such that hydrogel beads can be formed. In this aspect, polymeric coating agents, e.g. lipophilic polymers such as polyesters, PLA, PLGA, PCL and poly anhydrides etc. are preferred.

In some embodiments, the first emulsifying fluid comprises a coating agent as herein described (preferably a lipophilic and/or polymeric coating agent). In some embodiments, the coating fluid and the first emulsifying fluid have the same composition.

In some embodiments, the first emulsifying fluid comprises a coating agent, and thus a separate coating fluid is not required. In such embodiments, the emulsifying fluid from the bead formation step (e.g. the first emulsifying fluid) may be used as the carrier fluid for the bead coating step. Thus, the step of providing the mixture of carrier fluid (for the coating method) and hydrogel (micro)beads may comprise forming the hydrogel (micro)beads from the hydrogel precursor composition in the presence of the first emulsifying fluid.

In embodiments where the carrier fluid is the emulsifying fluid from the bead formation method, the resulting mixture of beads and (first) emulsifying fluid can be used directly in the coating method. For example, after formation of the beads (e.g. via gelling), the resulting mixture of beads in (first) emulsifying fluid can be contacted directly with the second emulsifying fluid, e.g. without separation from the first emulsifying fluid, collection or washing.

In a further embodiment, the present invention provides a method for forming coated hydrogel (micro)beads, said method comprising:
providing a hydrogel precursor composition;
providing a first emulsifying fluid, wherein the first emulsifying fluid is immiscible with the hydrogel precursor composition and comprises a coating agent;
forming a first emulsion, said first emulsion comprising the hydrogel precursor composition as a discontinuous phase and the first emulsifying fluid as a continuous phase;
forming hydrogel (micro)beads from the hydrogel precursor composition in the presence of the first emulsifying fluid;
providing a second emulsifying fluid, wherein the second emulsifying fluid is immiscible with the first emulsifying fluid;
forming a second emulsion, said second emulsion comprising the hydrogel (micro)beads and the first emulsifying fluid as a discontinuous phase and the second emulsifying fluid as a continuous phase; and
forming a coating on said hydrogel (micro)beads from the first emulsifying fluid.

In the methods herein described, the step of forming an emulsion comprises contacting the two phases at an intersection of two or more microfluidic channels. Thus, the step of forming the first emulsion may comprise contacting the hydrogel precursor composition with the first emulsifying fluid at an intersection of two (or more) microfluidic channels. Similarly, the step of forming the second emulsion may comprise contacting the mixture (i.e. the mixture of carrier/coating fluid and hydrogel beads, or the mixture of first emulsifying fluid and hydrogel beads) with the second emulsifying fluid at an intersection of two microfluidic channels.

Gelation of the droplets of hydrogel precursor composition results in beads suspended in the first emulsifying fluid. In embodiments where the first emulsifying fluid comprises a coating agent as herein described, the mixture of first emulsifying fluid and beads can be contacted with the second emulsifying fluid without the need for adding a separate coating fluid. Thus, the mixture of first emulsifying fluid and beads can be directly contacted with, e.g. directly reinjected into, the second emulsifying fluid, to form droplets of first emulsifying fluid (now functioning as carrier/coating fluid) which contain beads. In such embodiments, the first emulsifying fluid acts as the coating fluid, i.e. the carrier fluid is the first emulsifying fluid. In such embodiments, it is not necessary to mix the beads with the carrier/coating fluid as they are already present in it from the bead formation step.

For example, as outlined in Example 4 herein, hydrogel beads can be formed using, as the emulsifying fluid (first emulsifying fluid) for the first step, PLGA (a polymer intended for use as coating material) dissolved in DMC (hydrophobic solvent). As the hydrogel precursor composition and the PLGA+DMC fluid are immiscible, droplets of the hydrogel precursor composition are formed, which can be converted into hydrogel beads in the emulsifying fluid. The resulting mixture of hydrogel beads suspended in the first emulsifying fluid (PLGA+DMC fluid phase) can then be reinjected into a second microfluidic chip (with another intersection) and thus dispersed into a second emulsifying fluid (e.g. water with surfactant as in Example 5) to form droplets of PLGA+DMC in water, with the droplets containing a hydrogel bead from the bead formation step. Extraction of the DMC results in a coating (e.g. PLGA shell) around the hydrogel beads.

The second emulsification (i.e. the emulsification of the coating method) results in droplets of carrier fluid (e.g. coating fluid) in the (second) emulsifying fluid. At least some of the droplets contain a hydrogel bead. Converting the carrier fluid into a coating thus involves forming a coating, from the coating agent, around the bead. The step of forming a coating from the carrier fluid (e.g. coating fluid), or the coating agent it contains, can be achieved by any methods known in the art. In some embodiments, forming a coating from the coating fluid/agent can be achieved by removal of solvent (e.g. by solvent evaporation, solvent extraction, etc.), UV exposure, temperature, a chemical reaction or a combination thereof.

The droplets of carrier fluid which contain beads may be collected from the channel, chip or device in which the emulsion is formed (e.g. from the microfluidic chip) into a vial/container while still in emulsion with, i.e. dispersed in, the (second) emulsifying fluid, prior to formation of the coating.

In some embodiments, the coating is formed by evaporation of solvent from the carrier fluid. In some embodiments, the coating is formed by extracting solvent from the carrier fluid, e.g. by addition of ethanol/water/cosolvent,

As the carrier fluid and/or the coating agent is immiscible with the emulsifying fluid (i.e. the second emulsifying fluid), the carrier fluid (and thus the coating agent that it contains) will accumulate around the hydrogel bead and form the shell/coating.

Thus, in preferred embodiments, the coated hydrogel beads are formed, i.e. the coating is formed, in suspension in the emulsifying fluid (i.e. the second emulsifying fluid). The coated beads can then be separated from the emulsifying fluid, e.g. by filtration, centrifugation, or by evaporation of any solvent.

Typically, when the coating agent is, or comprises a pre-gel, UV exposure, temperature or chemical reactions can be used to form a coating from the carrier fluid, e.g. the coating fluid.

Typically, when the coating agent is not, and does not comprise, a pre-gel (e.g. when the coating agent is a polymer), solvent extraction or solvent evaporation can be used to form a coating from the carrier fluid, e.g. the coating fluid.

Solvent evaporation can be achieved by agitation (e.g. agitation overnight) of the loaded carrier/coating fluid droplets. In this context, “loaded” refers to the droplet of carrier fluid containing, i.e. being loaded with, a bead.

Solvent extraction can be achieved by addition of a suitable fluid for solvent extraction (e.g. a high volume of water or ethanol).

UV exposure can involve exposing the mixture of carrier/coating fluid and hydrogel bead in the (second) emulsifying fluid to a UV source. Suitable UV exposure wavelengths and periods are as described above in relation to bead formation. For example, UV exposure can involve a suitable UV exposure regime, for example one selected relative to any photoinitiator used. Typical UV exposure steps include exposure to 10-400 nm wavelength electromagnetic radiation, e.g. 100-400 nm (such as 100-315 (e.g. 100-280 nm) or 280-400 nm (e.g. 315-400 nm)), preferably 250-380 nm, 255-340 nm, 280-315 nm, or 365 nm. UV exposure can be for 10 seconds to 60 min, e.g. 30 seconds to 30 min, 45 seconds to 10 min, 1 min to 5 min, e.g. 3 min. UV exposure can be from 2 to 30 cm from the mixture of carrier/coating fluid and hydrogel bead, e.g. from 5 to 25 cm, 10 to 20 cm, 12 to 18 cm, 15 to 22 cm from the mixture of carrier/coating fluid and hydrogel bead.

Use of temperatures can involve exposing the loaded carrier/coating fluid droplets to temperatures as described above in relation to bead formation. Temperatures can be below the gelation temperature or above the critical gelation temperature, depending on the type of hydrogel used. For example, when ultra-low temperature melting agarose is used, a temperature of around 8° C. can be used, and when poloxamers (such as Pluronic® F127 or Pluronic® F68 are used) temperatures above the critical gelation temperature can be used, which depends on their concentration (for example, temperatures of around 30° C. for a 5 to 30% (e.g. 10 to 25%, 12 to 20%, 13 to 18%, preferably 15%) Pluronic® F127 or Pluronic® F68 precursor solution can be used).

Use of a chemical reaction as described above for bead formation can be used to form a coating from the coating fluid or coating agent. For example, use of a chemical reaction can involve incubation to let the reaction occur (e.g. incubation from 1 min to 6 hours, e.g. 5 min to 4 hours, 30 min to 2 hours, 45 min to 1 hour), depending on the concentration and strength of the functionalities. For example, for a Michael addition (e.g. with PEGs between thiol groups and “ene” groups [C═C bonds]), or a Diels Alder reaction (e.g. a Diels-Alder reaction between PEG with maleimide functionalities and furan functionalities), the chemical reaction can require a time from 1 min to 6 hours, e.g. 5 min to 4 hours, 30 min to 2 hours, 45 min to 1 hour to crosslink.

Use of pH to form the coating, e.g., via the means described above for hydrogel bead formation, may also be used to form the coating. For example PEG acrylate and PEG thiol reacts by Michael addition in minutes to hours at pH 7, but reacts in seconds to minutes at pH 8 and above. Use of a pH of 8 and above can be used to form the coating.

Ionic gelation, e.g. as described above, can also trigger formation of a coating from the coating fluid or coating agent. Ionic gelation, caused by diffusion of Ca²⁺ ions in the hydrogel can trigger formation of a hydrogel coating from a coating fluid which contains a hydrogel precursor as the coating agent. For example, ionic gelation can be used to rapidly (i.e. in less than 1 hour from exposure to Ca²⁺ ions, e.g. less than 45 min, less than 30 min, less than 15 min, less than 10 min or less than 5 min) trigger alginate crosslinking by ionic interactions.

The emulsifying fluid used in the method of coating hydrogel beads (i.e. the second emulsifying fluid) is immiscible with the carrier/coating fluid and/or the coating agent. Examples of suitable emulsifying fluids include any suitable hydrophilic or aqueous fluid if the carrier/coating fluid is lipophilic, e.g. if an organic solvent is present in the carrier/coating fluid, and any suitable lipophilic fluid, e.g. an organic solvent, if the carrier/coating fluid is hydrophilic, e.g. if a pre-gel is present in the carrier/coating fluid.

Examples of aqueous fluids include water (e.g. deionized water) and buffers such as phosphate buffer, PBS, DPBS (Dulbecco's Phosphate Buffered Saline). Aqueous fluids can contain surfactants (e.g. polyvinyl alcohol, PVA). An example of an aqueous fluid for use as emulsifying fluid in the coating step of the present invention is PVA in DPBS, e.g. 3% w/v PVA (9-10 k Mw 80% h) in DPBS.

Examples of organic solvents include those previously mentioned, e.g. oils, HFE (hydrofluoroethers), FC-40 (Fluorinert® oil), light mineral oil, olive oil, hexadecane, sunflower seed oil, castor oil, surfactants and combinations thereof. For example, the (second) emulsifying fluid may be a mixture of HFE and surfactant. Suitable solvents also include chloroform, dichloromethane (DCM), dimethylcarbonate (DMC), benzyl acetate, ethyl acetate, anisole, and mixtures thereof.

The channels as herein described are typically those suitable for microfluidic methods, e.g. they are sized such that the flow of fluid though them is laminar and that the flow of fluid is achieved by capillary action and/or pressure driven flows. Typically, channels will be microfluidic channels, e.g. in microfluidic devices or chips.

“Capillary action” will be understood to mean fluid flow in the absence of rotational motion or centripetal force applied to a fluid on a rotor or platform of the invention and is due to a partially or completely wettable surface.

Droplet production (e.g. hydrogel precursor droplets or hydrogel beads in carrier/coating fluid droplets) may be effected using pressure driven flows. Pressure driven flows can be achieved by pumps which generate pressure in a reservoir containing the fluid, which will push the fluid into the channels. Alternatively, droplet production can be achieved by syringe-pump-driven flows, i.e. those that cause a constant flow rate of the liquids injected in the microfluidic chip.

A type of microfluidic device suitable for use in the invention is a microfluidic chip.

Microfluidic devices and methods are well known in the art. However, certain microfluidic systems for performing the methods described herein are novel and thus form a further aspect of the invention. For example, the output of a first microfluidic chip can be directly connected to the input of a second microfluidic chip, e.g. to perform the direct coating method as described herein (i.e. where the first emulsifying fluid acts as the carrier fluid in the coating step).

Thus, a further aspect of the present invention provides a microfluidic system (e.g. for forming the coated hydrogel beads as described herein), the system comprising:
a first microfluidic chip and
a second microfluidic chip

wherein the output of the first chip is connected to the input of the second chip.

The output of the first chip may be directly connected to the input of the second chip.

The second chip is preferably downstream of the first chip.

Each chip may comprise at least two microfluidic channels which meet at an intersection. In the first chip, these channels may be adapted to contain, or do contain, the hydrogel precursor composition (in one channel) and the first emulsifying fluid (in another channel) described herein. In the second chip, these channels may be adapted to contain, or do contain, the mixture (i.e. of hydrogel beads and coating fluid or of hydrogel beads and first emulsifying fluid in one channel) and the second emulsifying fluid (in another channel) described herein.

The microfluidic chips or channels can be hydrophobic microfluidic chips, e.g. a PDMS (polydimethylsiloxane) hydrophobic microfluidic chip.

The microfluidic chips can be hydrophilic microfluidic chips.

The microfluidic chips can have a specific design to pack the hydrogel beads. Such a design would be known to a person skilled in the art.

In some embodiments of the present invention, the droplets/emulsions are formed using flow-focusing. The microfluidic chips can have a flow-focusing design. The microfluidic chip can have a nozzle with a width of from 15 to 80 μm, e.g. 20 to 75 μm, 25 to 60 μm, 30 to 55 μm, 35 to 50 μm, 40 to 45 μm, and/or a height of from 30 to 75 μm, e.g. 35 to 70 μm, 40 to 65 μm, 45 to 60 μm, 50 to 55 μm. The flow-focusing design can allow formation of a droplet of carrier/coating fluid containing one hydrogel bead, for example. For the bead formation step, the bead size can be controlled via the nozzle size. For the coating step, the nozzle size should be selected to be compatible with the size of the beads.

A microfluidic chip can comprise one or more channels and one or more valves controlling flow of fluid through the channels. In an alternative embodiment, a microfluidic chip can comprise one or more reservoirs, one or more channels connecting the reservoirs to each other, and one or more valves controlling flow of fluid through the channels.

Reservoirs can be part of the platform used with the microfluidic chip. Reservoirs can be glass vials pressurised by the pump that are linked to the microfluidic chip by plastic tubing (e.g. PTFE tubing). The tubing can be inserted in the microfluidic chip through inlets that can be manually punched (e.g. when the chip is made of silicon) and that allow transfer of the fluids from the reservoirs to the microfluidic channels embedded in the chip.

The fluids (i.e., the hydrogel precursor composition, the emulsifying fluids and the carrier/coating fluids) or the hydrogel beads (i.e. the hydrogel beads as herein described) can be injected or reinjected in the microfluidic chip using a needle.

Two or more channels in the microfluidic chip can meet at a junction linked to a further channel. At the junction, a dispersion can be formed, e.g. in the method described herein where a composition or fluid is brought into contact with a composition or fluid in which it is immiscible. An example is the dispersion formed when the hydrogel precursor composition is brought into contact with the emulsifying fluid during preparation of a hydrogel bead as herein described. A further example is the dispersion formed when the mixture of hydrogel beads in carrier/coating fluid is brought into contact with the emulsifying fluid during coating of a hydrogel bead as herein described.

The channels in the microfluidic chip can be engraved in the substrate of the chip (e.g. a PDMS chip (a silicon chip), a PMMA chip, a glass chip) by different methods such as etching, soft lithography moulding, and hot embossing.

Different geometrical features can also be included in the design to functionalize the chip such as filters, valves, mixing features, sorting features, traps and intersections (e.g. the junctions mentioned herein) to generate droplets.

A microfluidic chip can further comprise a pump to generate pressure in a reservoir or a syringe pump to inject fluids into the channels.

A microfluidic chip can further comprise inlet and outlet ports. An inlet port may connect the reservoir and the chip. An outlet port may connect the chip to the collecting vial.

The droplets formed in the microfluidic chip can be collected out of the chip into a container such as a vial, e.g. in the emulsion. The vial can then be exposed to UV, temperature, ionic gelation or a chemical reaction (e.g. for the formation of the gel bead, or the formation of the coating). The solvent in the vial could alternatively or additionally be extracted overnight, e.g. by low agitation. Removal of solvent can be used when a large volume of solvent is used to collect the droplets out of the chip.

For all the methods described herein, the fluids used (i.e., the hydrogel precursor composition, the emulsifying fluids and the carrier/coating fluids) can be selected to exhibit specific properties. In one embodiment, the viscosity of these fluids is from 0 to 200 mPa·s, e.g. 5 to 150 mPa·s, 10 to 100 mPa·s, 20 to 80 mPa·s, or less than 180, less than 120, less than 75, less than 50 mPa·s. In one embodiment, the density of these fluids is from 0 to 3 g/cm³, e.g. 0.5 to 2.5 g/cm³, 1 to 2 g/cm³. In one embodiment, the surface tension of these fluids is from 0 to 100 mN/m, e.g., 5 to 80, 10 to 75, 20 to 60, 30 to 50 mN/m. The examples of the fluids provided herein may exhibit one or more of these properties.

The emulsifying fluid and carrier/coating fluid preferably have an interfacial tension of 10 to 50 mM/m, preferably 20 to 40 mM/m, e.g. 22 to 38 mN/m, 25 to 35 mN/m, 28 to 32 mN/m or 30 to 33 mN/m. “Interfacial tension” is defined as the work which must be expended to increase the size of the interface between two adjacent phases which do not mix completely with one another. The two adjacent phases can be liquid/liquid or liquid/solid.

The various fluids described herein are preferably in liquid form. Thus, the first emulsifying fluid, the hydrogel precursor composition, the carrier/coating fluid and/or the second emulsifying fluid, are preferably liquids.

The steps of forming a hydrogel bead and coating a hydrogel bead as described herein may be carried out in the same or different microfluidic device or chip. In one embodiment, two separate microfluidic devices/chips are used to form a hydrogel bead and coat the hydrogel bead as herein described. In some embodiments, the bead is formed and coated in the same system or device.

The step of forming a hydrogel bead may be separate from the step of coating the hydrogel bead. Without being bound by theory, this can remove the need for specific surface coatings and can enable more control over the core and shell size than could be achieved in a double emulsion process.

Channel sizes are optimally determined by specific applications and by the amount of and delivery rates of fluids required for the method of the invention. Channel sizes (e.g. the diameter of the interior cross section) can range from 0.1 μm to about 1 mm, e.g. 0.5 μm to about 500 μm, 1 μm to about 800 μm, 2 μm to 600 μm, 5 μm to 100 μm, 10 μm to 75 μm. The length of the channels can be from 1 μm to 10 cm, e.g. 10 μm to 5 cm, 100 μm to 3 cm. The channel cross section can be trapezoid, rectangular, square, circular or other geometric shapes as required. If the cross section is not uniform in diameter, the interior cross section stated above is for the largest cross section measurement.

The flow rate of the fluids in the channels is typically from 0 to 5 mL/hr, e.g. from 0.1 to 4 mL/hr, 0.5 to 3 mL/hr, 1 to 2 mL/hr. The flow rates can be adapted to achieve the desired diameter of the beads and/or coating material.

In some embodiments, droplets (e.g. droplets of hydrogel precursor composition or droplets of carrier/coating fluid) are formed at a throughput of 50 to 1000, e.g. 100 to 500, or 200 to 400 droplets per second.

The channels, e.g. the junction of the first and second inlet channel is sized such that when the fluids meet, a dispersion or emulsion is formed. The size of the nozzle of the junction (i.e. the part of the junction where the droplets are formed after the intersection) can influence the size of the droplet that is formed. The size can range from 20 μm wide to 500 μm wide, e.g. 20 μm wide to 300 μm wide, 25 μm wide to 250 μm wide, 30 μm wide to 100 μm wide, 35 μm wide to 80 μm wide, 40 μm wide to 70 μm wide, 50 μm wide to 60 μm wide, preferably 20 μm wide to 80 μm wide, 25 μm wide to 75 μm wide, 30 μm wide to 70 μm wide, 35 μm wide to 65 μm wide, 40 μm wide to 60 μm wide, 45 μm wide to 55 μm wide or 40 μm wide to 50 μm wide.

Typical droplet diameters are 10 μm to 100 μm, e.g. 20 μm to 80 μm, 25 μm to 60 μm, 30 μm to 50 μm, e.g. around 40 μm.

The relative ease and simplicity of the methods described herein lends them to use in hospital pharmaceutical compounding environments, e.g. to deliver personalised medicines. The concentration of the encapsulated agent in the hydrogel core is flexible and the release kinetic tunability of the shell (which can be determined via choice of coating agent) is beneficial for developing controlled release systems for currently unavailable dosages of drugs. Some encapsulated agents, e.g. delayed release dyes, may have non-medical uses.

Coated hydrogel beads (e.g. coated hydrogel beads formed using the methods disclosed herein) form a further embodiment of the invention and can comprise a hydrogel bead “core” surrounded by a coating or “shell” (e.g. a polymer or hydrogel coating). The hydrogel bead “core” of the coated hydrogel beads can encapsulate an encapsulated agent.

Prior to coating, the hydrogel beads disclosed herein can range in size from 2 to 500 μm, e.g. 5 to 200 μm, 10 to 150 μm, 20 to 100 μm or 20 to 80 μm, 50 to 75 μm across a largest dimension.

The beads of the present invention (which includes those formed by the method of the present invention) comprise a hydrogel bead (e.g. the “core”), surrounded by a coating (e.g. the “shell”). The hydrogel core may comprise, consist of, or consist essentially of hydrogels formed from the hydrogel precursor compositions (and the optional further components, e.g. additives, encapsulated agents etc.) described herein. Examples of hydrogel materials include: cross-linked hydrophilic polymers, polysaccharides (e.g. natural polysaccharides), proteins (e.g. natural proteins), monomers (e.g. synthetic monomers) or polymers formed therefrom, polyacrylates, PEG (polyethylene glycol) derivatives, poloxamers, Poly(N-isopropylacrylamide) or PVA (polyvinyl alcohol), e.g. those mentioned herein, or combinations thereof. PEG beads are especially preferred.

Similarly, the coating or shell may comprise, consist of, or consist essentially of coatings formed from the coating agents described herein. Examples of coating materials include polymers such as PLGA. Especially preferred are polymer coatings, e.g. lipophilic polymer coatings as described herein.

The hydrogel bead of, or formed by the methods of, the present invention may be spherical or non-spherical, preferably substantially spherical.

The coating surrounding the hydrogel bead can be substantially uniform in thickness. The coating typically covers substantially all of the bead surface, e.g. the bead is fully coated.

The hydrogel bead coating as herein described may be, or comprise, a polymer and/or a hydrogel.

The coating surrounding the hydrogel bead can have a thickness of from 0.01 to 200 μm, e.g. 0.02 to 100 μm, 0.05 to 50 μm, 0.1 to 20 μm, 0.5 to 10 μm or 1 to 10 μm, 0.75 to 5 μm, 1 to 3 μm.

The coated hydrogel beads (e.g. coated hydrogel beads formed using the methods disclosed herein) can range in size from 2 micrometers to 1000 micrometers across a largest dimension (which, in the case of spherical coated hydrogel beads, is the diameter). In some embodiments, the coated hydrogel beads can be, for example, 10-1000, 40-1000, 20-900, 30-800, 40-700, 50-600 or 50 to 100, 60-500 micrometers across a largest dimension.

The coated hydrogel beads can be, for example, about 50 micrometers, about 100 micrometers, about 150 micrometers, about 200 micrometers, about 250 micrometers, about 300 micrometers, about 350 micrometers, about 400 micrometers, about 450 micrometers, about 500 micrometers, about 550 micrometers, about 600 micrometers, about 650 micrometers, about 700 micrometers, about 750 micrometers, about 800 micrometers, about 850 micrometers, about 900 micrometers, about 950 micrometers, and about 1000 micrometers across a largest dimension.

Coated hydrogel beads as discussed herein can be highly uniform in size. For example, the coefficient of variance (COV) for the coated hydrogel bead diameter can range from about 2% to about 9%, e.g. 3% to 8%, 4% to 6%, for example a COV of about 2%, a COV of about 2.5%, a COV of about 3%, a COV of about 3.5%, a COV of about 4%, a COV of about 4.5%, a COV of about 5%, a COV of about 5.5%, a COV of about 6%, a COV of about 7%, a COV of about 8%, and a COV of about 9%. Coated hydrogel beads as discussed herein have a high degree of roundness. For example, in one embodiment, the roundness is greater than 0.95 for all coated hydrogel beads.

In some embodiments, encapsulated agents can be encapsulated in the coated hydrogel beads (e.g. coated hydrogel beads formed by the methods disclosed herein) at a concentration of up to 80 wt %, e.g. 5 to 80 wt %, 5 to 75 wt %, 10 to 60 wt %, 20 to 50 wt %, 30 to 40 wt %, or 25 to 65 wt %, preferably, up to 5 wt %, 1 to 4 wt %, 1 to 3 wt %.

The coated hydrogel beads (e.g., coated hydrogel beads formed using the methods disclosed herein) with one or more encapsulated agents encapsulated in the hydrogel (e.g. in the “hydrogel core”), can be considered to be a controlled-release composition or delivery system. Controlled-release compositions comprising the coated beads as herein described thus form a further aspect of the present invention. Use of the coated hydrogel beads as herein described in a controlled-release composition forms a further aspect of the present invention. The controlled release compositions may be used for medical or non-medical uses. Use of the methods as herein described in the manufacture of controlled-release compositions form a further aspect of the present invention. “Controlled-release” refers to any composition where an encapsulated agent or drug delivery profile is modified from immediate encapsulated agent, or drug, release. Non-limiting examples of controlled release include sustained release, delayed release, pulsatile release, etc. Controlled-release can include dosing that occurs over a period of at least 1-2 hours, and can often last for a period of days or even weeks. A 12 hour or 24 hour dosing release profile is common.

Controlled-release compositions and or the coated beads as herein disclosed can include a therapeutically effective amount of one or more encapsulated agents.

Controlled-release compositions and/or the coated beads as herein disclosed can be used in microparticulate delivery systems, for the production of complex delayed drug delivery systems, for encapsulating vaccines for delayed delivery, for producing tailored drug delivery systems with specific release kinetics, for encapsulating cosmetics, and for relaying release of foodstuff (e.g. delayed release of flavours, vitamins or antioxidants).

References herein to “comprising” should be understood to include “consisting of” and “consisting essentially of” and may be substituted for such where relevant.

It will be appreciated that the disclosed methods may take advantage of any of the materials described above in relation to the products. Likewise, features disclosed in relation to the methods are also relevant to the products.

Certain preferred embodiments of the invention will now be described by way of the following non-limiting examples and with reference to the accompanying drawings in which:

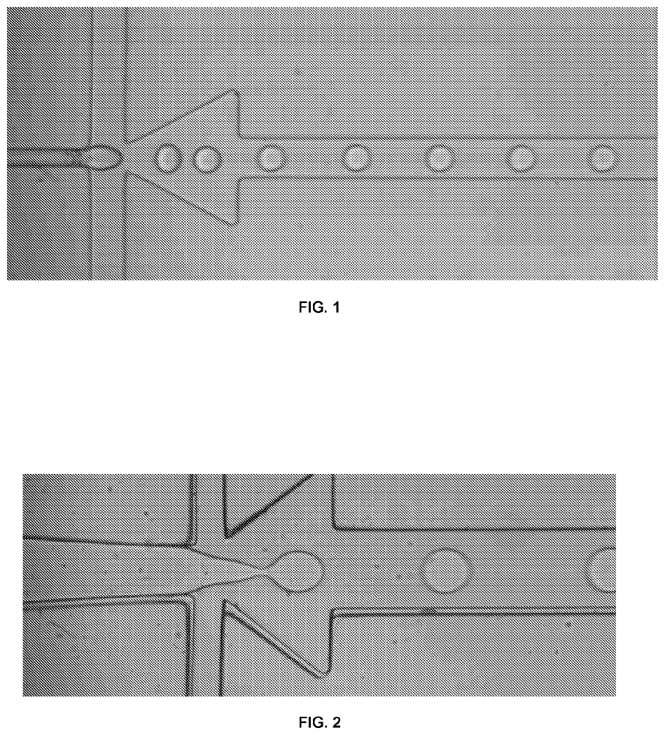

FIGS. 1 and 2 show a hydrogel precursor composition being injected from the left into a first emulsifying fluid and forming droplets, in accordance with the microfluidic method of the present invention and Example 4.

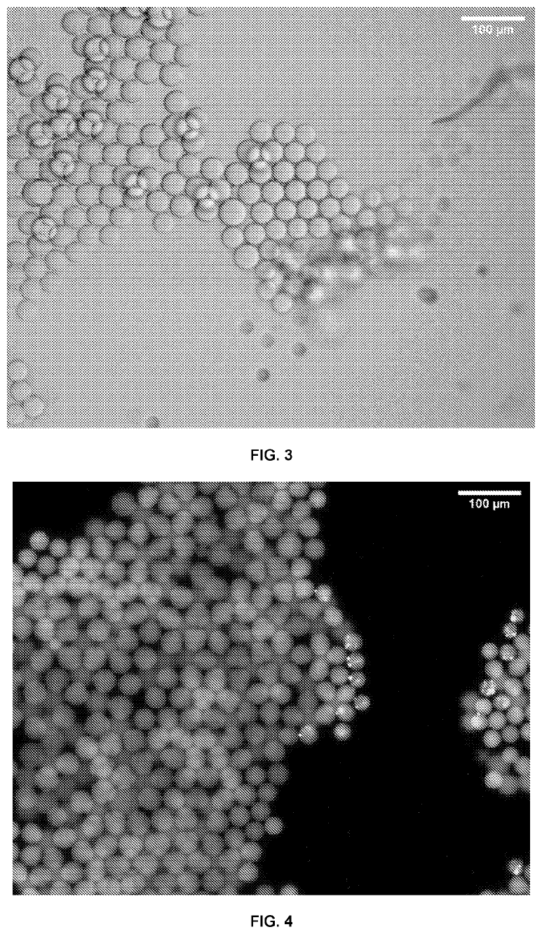

FIGS. 3 and 4 show hydrogel beads formed in Example 4 using the microfluidic method of the present invention, after gelation, and prior to coating. FIG. 3 shows the bright-field (normal light) image of the beads. FIG. 4 shows the emission fluorescence coming from a dye encapsulated inside the hydrogel beads when excited by a laser.

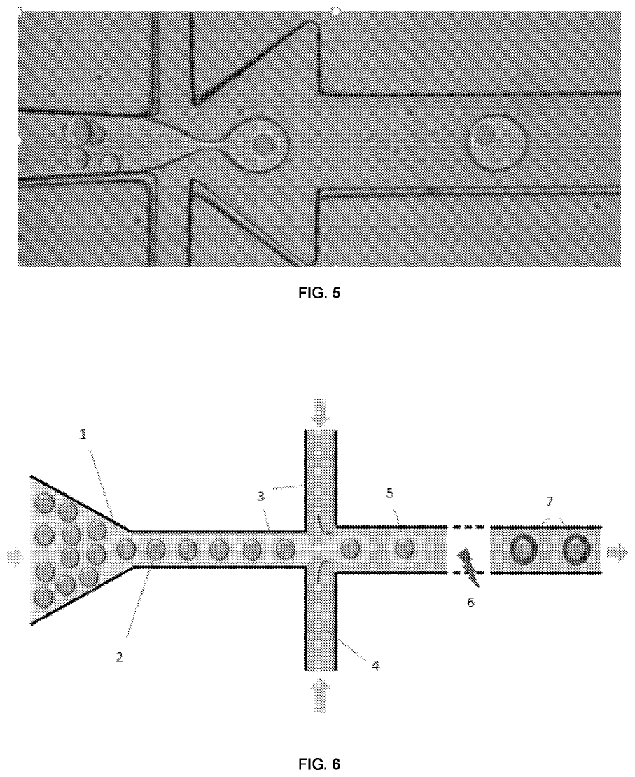

FIG. 5 shows injection of a mixture of carrier fluid and hydrogel beads from the left into second emulsifying fluid, and the formation of droplets of carrier fluid containing a hydrogel bead, in accordance with the microfluidic method of the present invention and Example 5.

FIG. 6 is a schematic of the method of coating hydrogel beads. A carrier/coating fluid as herein described (1) which contains a hydrogel bead as herein described (2) can pass through a microfluidic channel (3). An emulsifying fluid (4) (referred to also as the second emulsifying fluid herein) that is immiscible with the coating fluid (1) can be brought into contact with the coating fluid (1) to form droplets of coating fluid which contain hydrogel beads (5). In one embodiment, as shown, the emulsifying fluid is brought into contact with the coating fluid (1) at a junction of microfluidic channels (3). The droplets of coating fluid which contain the hydrogel beads (5) are then subjected to a coating forming step (6). The step of forming the coating can be achieved by any methods known in the art, such as solvent evaporation, solvent extraction, UV exposure, temperature, a chemical reaction or a combination thereof. When the coating forming step has occurred, a coated hydrogel bead (7) is formed.

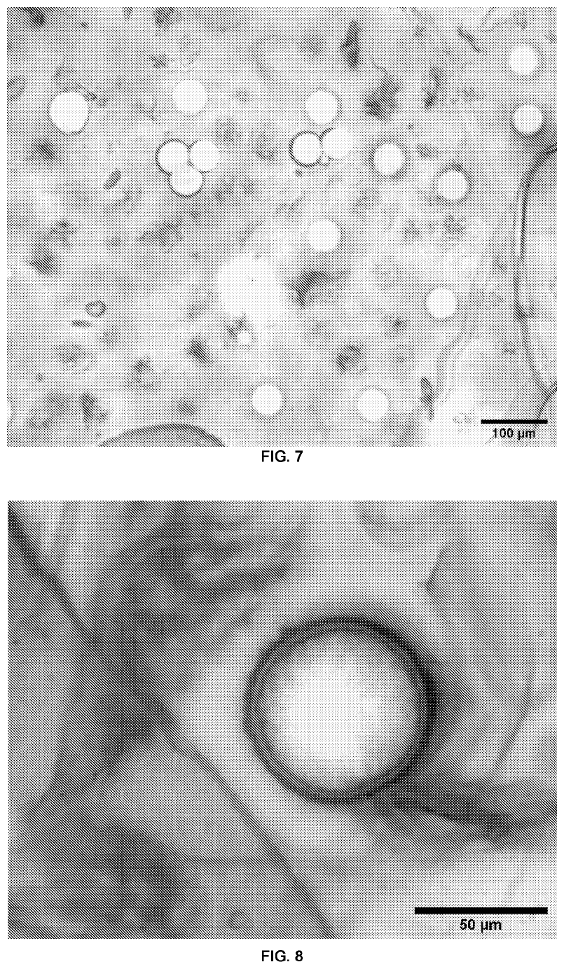

FIG. 7 shows coated hydrogel beads formed in Example 5 after evaporation of solvent from the carrier fluid.

FIG. 8 shows a coated hydrogel bead formed via Example 5 in accordance with the microfluidic method of the present invention.

FIGS. 7 and 8 show the emission fluorescence from the dye encapsulated inside the hydrogel beads cores when excited by laser. These images are overlays of the bright-field to show the coating (grey layer around the fluorescent cores) and the core (lighter fluorescent signal due to dextran RhodaminB trapped inside)

### EXAMPLE 1—METHOD OF FORMING A HYDROGEL BEAD

A hydrogel precursor solution is prepared by mixing 8-arm PEG norbornene (example concentration: 20% w/v of solution), LAP (example concentration: 5 mM) and dithiol linker (e.g. dithiothreitol) in equimolar quantities with the PEG norbornene in an aqueous solvent (example: deionized water). An encapsulated agent can be added at this step.

This solution (hydrogel precursor composition) is then brought into contact with HFE with surfactant as emulsifying fluid, using a PDMS (polydimethylsiloxane, a silicon material) hydrophobic microfluidic chip with a flow focusing design (example size: nozzle of 30 μm width 50 μm height), to form a droplet of hydrogel precursor composition around 40 μm diameter size. The droplets are collected out of the chip in a larger volume of HFE in a vial and crosslink by exposing the vial and droplets to a UV source (365 nm wavelength, 3 min, placed at 10 cm of the vial). Hydrogel beads are formed.

### EXAMPLE 2—METHOD OF MAKING A COATED HYDROGEL BEAD (GEL COATING)

Hydrogel beads previously formed are re-suspended in the suitable coating fluid, for example another hydrogel mixture made of 8-arm PEG acrylate and 8-arm PEG thiol in equimolar concentrations of acrylate and thiol. This mixture is reinjected in a hydrophobic PDMS microfluidic chip with a specific design to pack the hydrogel beads and a flow focusing design to form the droplet of coating fluid loaded with one hydrogel bead (flow focusing nozzle geometry superior to the hydrogel bead size, example: 50 μm width, 60 μm depth). HFE with surfactant is used as the (second) emulsifying fluid and flow rates are adapted to achieve the desired diameter of coating material. The droplets containing hydrogel beads are collected out of the chip in a vial and let to crosslink overnight (Michael addition between acrylate and thiol) to form core shell hydrogel structure with the active agent contained in the core geometry.

### EXAMPLE 3—METHOD OF MAKING A COATED HYDROGEL BEAD (POLYMER COATING)

Hydrogel beads previously formed are re-suspended in a solution of PLGA (example 7-17 kDa molecular weight, 50:50 lactic:glycolic ratio) in DMC (dimethyl carbonate, a solvent) as the coating fluid. The mixture is reinjected in a hydrophilic microfluidic chip with a specific design to pack the hydrogel beads and a flow focusing design to form the droplet of coating fluid loaded with one hydrogel bead (flow focusing nozzle geometry superior to the hydrogel bead size, example: 50 μm width, 60 μm depth). Water with PVA (polyvinyl alcohol, a surfactant) is used as the (second) emulsifying agent and flow rates are adapted to achieve the desired diameter of coating material. The droplets containing hydrogel beads are collected out of the chip in a vial with large volume of water and DMC is extracted overnight by low agitation in the large volume of water. Hydrogel core-PLGA shell particles are obtained with the active agent contained in the core.

### EXAMPLE 4—METHOD OF FORMING A HYDROGEL BEAD

Materials:
Inner Phase (encapsulated agent): Dextran-RhodaminB (3.2 mg/mL in Pre-gel material)
Pregel Material: 5% w/v PEG-Norbornene 8-arms (20 mM [NB] groups), 40 mM DTT (80 mM [SH] group) 1% w/v LAP initiator
Outer Phase (emulsifying fluid 1): 10% w/v PLGA R502 (7-17 k Mw, 50;50, ester cap) in DMC (dimethylcarbonate)
Microfluidic chip: Flow focusing, PDMS, 50 μm*25 μm nozzle

Method:

A hydrogel precursor composition was prepared by mixing the pre-gel material with the inner phase (encapsulated agent). The precursor composition was then brought into contact with the outer phase (emulsifying fluid 1, PLGA+DMC), in the microfluidic chip mentioned above. FIG. 1 shows droplets were formed at a throughput of ˜300 droplets per second.

The droplets were collected from the microfluidic chip into a vial/container while still in suspension with the emulsifying fluid (i.e. inside an Eppendorf in the PLGA+DMC phase) and then cross-linked to polymerise the hydrogel by exposure to 365 nm UV, for 3 minutes at maximum intensity to form hydrogel beads. The beads formed are shown in FIGS. 3 and 4. FIG. 4 shows the emission fluorescence coming from the dye encapsulated inside the hydrogel beads when excited by a laser.

### EXAMPLE 5—METHOD OF COATING A HYDROGEL BEAD (REINJECTION/SECOND EMULSIFICATION)

Materials:
Reinjection Phase (mixture of beads and carrier fluid): Hydrogel beads in 10% PLGA in DMC (from Example 4 first emulsification)
Outer Phase (emulsifying fluid 2): 3% w/v PVA (9-10 k Mw 80% h) in DPBS
Microfluidic chip: Flow focusing, PDMS, 100 μm*80 μm nozzle

Method:

The mixture of hydrogel beads in 10% PLGA in DMC formed in Example 4 was brought into contact with the outer phase (emulsifying fluid 2), in the microfluidic chip mentioned above. In this case, the 10% PLGA in DMC which functioned as an emulsifying fluid in the bead formation method of Example 4 is now being employed as the carrier fluid for the coating step. Droplets of carrier fluid were formed around beads as shown in FIGS. 5 and 6. After extracting the solvent (DMC) from the carrier fluid, a PGLA coating was formed around the hydrogel bead. Thus, core-shell particles were formed from hydrogel core template, as shown in FIGS. 7 and 8. FIGS. 7 and 8 show the emission fluorescence from the dye encapsulated inside the hydrogel beads cores when excited by laser. These images are overlays of the brightfield to show the coating (grey layer around the fluorescent cores) and the core (lighter fluorescent signal due to dextran RhodaminB trapped inside).

Examples 4 and 5 demonstrate that, by selecting the same (or similar) composition for the emulsifying fluid in the bead formation step (i.e. emulsifying fluid 1) as is to be used as the coating fluid in the coating step, it is not necessary to collect and wash the beads following polymerisation/gelation. The beads may be formed from droplets while still in the first emulsifying fluid, and the resultant mixture of first emulsifying fluid and beads can be injected unto the second emulsifying fluid to form droplets of first emulsifying fluid (now functioning as coating fluid) which contains beads.

1. A method for forming coated hydrogel microbeads, said method comprising:
forming an emulsion comprising a carrier fluid and hydrogel microbeads as a discontinuous phase, and an emulsifying fluid as a continuous phase; and
forming a coating on said hydrogel microbeads from the carrier fluid;
wherein said carrier fluid comprises a coating agent and the emulsifying fluid is immiscible with the carrier fluid.

2. The method of claim 1, said method comprising, prior to forming the emulsion comprising the carrier fluid and hydrogel microbeads as a discontinuous phase, providing a mixture of the carrier fluid and hydrogel microbeads.

3. The method of claim 2 where said step of providing the mixture comprises forming said hydrogel microbeads via a method comprising:
forming an emulsion, e.g. a first emulsion, comprising a hydrogel precursor composition as a discontinuous phase and a first emulsifying fluid as a continuous phase; and
forming hydrogel microbeads from the hydrogel precursor composition;
wherein the first emulsifying fluid is immiscible with the hydrogel precursor composition.

4. The method of claim 2 or claim 3 wherein the carrier fluid is a coating fluid and the step of providing the mixture of carrier fluid and hydrogel microbeads comprises combining the hydrogel beads with the coating fluid.

5. The method of claim 3 wherein the first emulsifying fluid is used as the carrier fluid, e.g. wherein the step of providing the mixture of carrier fluid and hydrogel microbeads comprises forming the hydrogel microbeads from the hydrogel precursor composition in the presence of the first emulsifying fluid.

6. The method of claim 5, said method comprising:
providing a hydrogel precursor composition;
providing a first emulsifying fluid, wherein the first emulsifying fluid is immiscible with the hydrogel precursor composition and comprises a coating agent;
forming a first emulsion, said first emulsion comprising the hydrogel precursor composition as a discontinuous phase and the first emulsifying fluid as a continuous phase;
forming hydrogel microbeads from the hydrogel precursor composition in the presence of the first emulsifying fluid;
providing a second emulsifying fluid, wherein the second emulsifying fluid is immiscible with the first emulsifying fluid;
forming a second emulsion, said second emulsion comprising the hydrogel microbeads and the first emulsifying fluid as a discontinuous phase and the second emulsifying fluid as a continuous phase; and
forming a coating on said hydrogel microbeads from the first emulsifying fluid.

7. The method of any preceding wherein the step of forming an emulsion comprises contacting the two phases at an intersection of two microfluidic channels.

8. The method of any preceding claim wherein the coating is a polymer coating.

9. The method of any preceding claim wherein the carrier fluid comprises a lipophilic polymer and a hydrophobic solvent.

10. The method of any preceding claim wherein the hydrogel precursor composition or the hydrogel microbead comprises an encapsulated agent, preferably an encapsulated agent selected from pharmaceutical compounds or compositions, nutritional supplements, flavours, minerals, vitamins, small molecule drugs, proteins, therapeutic viruses, vaccines, therapeutic antibodies, nutritional supplements, carotene, biomarkers, tracer dyes, fluorescent dyes and combinations thereof.

11. A method for forming a coated hydrogel microbead (e.g. a method as claimed in any preceding claim) wherein the hydrogel microbead bead is coated via, and optionally also formed via, microfluidics.

12. A coated hydrogel microbead formed by the method of any preceding claim.

13. A coated hydrogel microbead (e.g. as claimed in claim 12) with a size in the range of 40 micrometres to 1000 micrometres across the largest dimension, and a coefficient of variance of between 2 and 9%, preferably wherein said coated hydrogel microbead has one or more encapsulated agents encapsulated in the hydrogel.

14. A controlled release composition comprising a coated hydrogel microbead according to claim 12 or claim 13.

15. A microfluidic system (e.g. for forming coated hydrogel microbeads by the method of any one of claims 1 to 11), the system comprising:
a first microfluidic chip; and
a second microfluidic chip;
wherein the output of the first chip is connected to the input of the second chip.
